(19) United States
(12) Patent Application Publication
(10) Pub. No.: US 2011/0287091 A1
(43) Pub. Date: Nov. 24, 2011

(54) CHRONOTHERAPEUTIC PHARMACEUTICAL COMPOSITION

(75) Inventors: Sanjay Boldhane, Maharashtra (IN); Shripad Jathar, Maharashtra (IN); Maneesh Nerurkar, Maharashtra (IN)

(73) Assignee: Abbot Healthcare Private Limited, Chembur (IN)

(21) Appl. No.: 13/145,765

(22) PCT Filed: Jan. 21, 2010
(86) PCT No.: PCT/IN10/00035

§ 371 Date: Aug. 9, 2011

(30) Foreign Application Priority Data
Jan. 22, 2009 (IN) 140/MUM/2009

(51) Int. Cl. A61K 9/48; A61K 9/00; A61K 9/28; A61P 1/04; A61K 31/192; A61P 29/00; A61P 9/10; A61P 9/12; B29C 43/02; A61K 9/16

(52) U.S. Cl. 424/451; 264/123; 424/400; 424/474; 424/490; 514/569

(57) ABSTRACT

The present invention relates to chronotherapeutic pharmaceutical compositions and a method of preparing the same. The composition comprises of at least one active ingredient, a pH independent agent and a hydrophilic agent. The active ingredient in the composition is coated with the pH independent agent. The composition provides a dual controlled release system, which aids in an initial lag time of 4-6 hours and controlled release of the active ingredient up to 24 hours.

### FIELD OF INVENTION

The present invention relates to chronotherapeutic pharmaceutical compositions and a method of preparing them.

### BACKGROUND OF THE INVENTION

Oral controlled release has been the most popular drug delivery system for obvious advantages of oral route of drug administration. It ensures sustained action of drug release over a prolonged period of time maintaining plasma concentrations in the therapeutic window.

Certain disease conditions demand release of drug after a lag time. The drug should not be released for the first 2 to 6 hours. After this lag time the drug should be released either in pulses or in an extended release manner so as to achieve the desired therapeutic action.

The conditions, which demand such release pattern include:

a) Physiological functions that follow circadian rhythm and cause a rise and fall in hormones like renin, aldosterone and cortisol etc.
b) Diseases that display chronopharmacological dependence like rheumatoid arthritis, gastrosophageal reflux disease, bronchial asthma, myocardial infarction, angina pectoris, hypertension etc.

These types of drug delivery systems, which release bioactive agents at a rhythm that ideally matches the biological requirement of a given disease therapy are called chronotherapeutic drug delivery systems, they include time-controlled and site-specific drug delivery systems.

Researchers have now found that the timing for the taking of a medicine can affect the way the human body responds to the medicine. The science of treating the human body taking into account the natural circadian variation is Chronotherapeutics. Chronotherapeutics relies on the practice of delivering the correct amount of medication to the correct site of action at the most appropriate time period for the particular disease or condition.

The major objective of chronotherapy for indications such as rheumatoid arthritis, gastric acid secretion, asthma and cardiovascular diseases is to deliver the drug in the desired concentrations during the time of greatest need and in lesser concentrations when the need is less. Our circadian rhythm is based on sleep-activity cycle and is influenced by our genetic makeup and thereby affects our bodies' function throughout day and night (24-hour period).

Arthritis is a group of conditions involving damage to the joints of the body. Arthritis is the leading cause of disability in people older than fifty-five years. There are different forms of arthritis; each has a different cause. The most common form of arthritis is osteoarthritis (degenerative joint disease) is a result of trauma to the joint, infection of the joint, or age. Emerging evidence suggests that abnormal anatomy might contribute to the early development of osteoarthritis. Other arthritis forms are rheumatoid arthritis and psoriatic arthritis. Septic arthritis is caused by joint infection. Gouty arthritis is caused by deposition of uric acid crystals in the joint, causing inflammation.

Rheumatoid arthritis (RA) is a chronic, systemic autoimmune disorder that most commonly causes inflammation and tissue damage in joints (arthritis) and tendon sheaths, together with anemia. It can also produce diffuse inflammation in the lungs, pericardium, pleura, and the sclera of the eye, and also nodular lesions, most common in subcutaneous tissue under the skin. It can be a disabling and painful condition, which can lead to substantial loss of functioning and mobility. It is diagnosed chiefly on symptoms and signs, but also with blood tests (especially a test called rheumatoid factor) and X-rays. Diagnosis and long-term management are typically performed by a rheumatologist, an expert in the diseases of joints and connective tissues. It is the clinical experience of rheumatologists that RA patients particularly experience joint pain, joint swelling, morning stiffness and functional disability in the early morning hours, with respect to arthritis, chronobiological patterns have been observed with arthritis pain. People with osteoarthritis tend to have less pain in the morning and more at night, whereas for people with rheumatoid arthritis the pain usually peaks in the morning and decreases as the days wears on. Past animal studies have shown that joint inflammation in rats fluctuates over a 24 hour period, this observation is supported by patients and physician.

Potential drug candidates for treatment of arthritis include NSAIDs and corticosteroids. Preferably the dosages should be timed to ensure that the highest blood levels of the drug coincide with peak pain. For osteoarthritis the optimal time for an NSAID would be around noon or mid afternoon. For rheumatic arthritis the optimal time for an NSAID to be taken is after the evening meal.

US20050276853 assigned to Penwest pharmaceuticals is directed to a chronotherapeutic pharmaceutical formulation comprising a core of active ingredient and a delayed release compression coating comprising a natural or synthetic gum applied onto the surface of core.

U.S. Pat. No. 6,346,268 assigned to Duramed pharmaceuticals is directed to a depot drug formulation including active ingredient and three-component release rate controlling matrix composition. The three components of matrix composition used in the invention are pH dependent gelling polymer as alginate component, an enteric polymer component and a pH independent gelling polymer

US20060099260 assigned to Biokey Inc. is directed to a pharmaceutical composition comprising a core comprising bupropion and, a coating comprising a pharmaceutically acceptable pH independent polymer and a surfactant

It is now considered desirable by those skilled in the art to provide the oral controlled release compositions that is adaptable to deliver the drug(s) of class NSAIDs such that the release rates and drug plasma profiles can be matched to physiological and chronotherapeutic requirements. In spite of existing prior arts mentioned above there is still need for an invention that is better in controlling symptoms of arthritis and convenient to manufacture, is economical in process and which meets the need for chronotherapeutic drug delivery system.

### SUMMARY OF THE INVENTION

An object of the invention is to provide a chronotherapeutic pharmaceutical composition that is effective in controlling diseases that show chronopharmacological dependence.

An aspect of the present invention relates to a chronotherapeutic pharmaceutical composition comprising of at least one active ingredient coated with agents or polymers, which are pH independent. The composition further comprises of hydrophilic agents that are mixed with the coated active ingredient. The active ingredient is released initially after a certain lag time followed by controlled release of the active ingredient as per the body's circadian rhythm. The lag time of the delayed extended release active ingredient is 4-6 hours thereby followed by the controlled release of the active ingredient over a time period of up to 24 hours. The composition is further enterically coated by means of pH dependent polymers.

Another aspect of the invention comprises of a process to prepare a chronotherapeutic pharmaceutical composition, which comprises of an active ingredient, a pH independent agent and a hydrophilic agent. The process comprises of coating the active ingredients with pH independent agent. The coated active ingredients are then blended with hydrophilic agents and compressed into tablets. The compressed tablets are further enterically coated to provide the chronotherapeutic composition.

### BRIEF DESCRIPTION OF THE DRAWING

FIG. 1 is a graph showing the dissolution profile in accordance with Table 1.

### DETAILED DESCRIPTION OF THE PREFERRED EMBODIMENTS

According to an embodiment of the present invention, a chronotherapeutic pharmaceutical composition comprises of at least one active ingredient, a pH independent agent and a hydrophilic agent. Only the active ingredient is coated with the pH independent agent or pH independent polymer. The hydrophilic agent forms a matrix around the coated active ingredient. The concentration of the active ingredient is 1 mg to 1000 mg. The composition provides an initial lag time of up to 4-6 hours followed by controlled release of the active ingredient up to 24 hours.

The active ingredient of the chronotherapeutic pharmaceutical composition is from the class of Non-steroidal anti-inflammatory drug (NSAID). The NSAIDs are selected from the group comprising of naproxen, lornoxicam, dilcofenac, ibuprofen and salts thereof. Preferably, naproxen sodium is the NSAID used for the chronotherapeutic pharmaceutical composition.

Naproxen is a propionic acid derivative related to the arylacetic acid group of nonsteroidal anti-inflammatory drugs. The chemical names for Naproxen and Naproxen sodium are “(S)-6-methoxy-a-methyl-2-naphthaleneacetic acid” and “(S)-6-methoxy-α-methyl-2-naphthaleneacetic acid, sodium salt”, respectively. Naproxen and Naproxen sodium have the following structures, respectively represented by formula I:

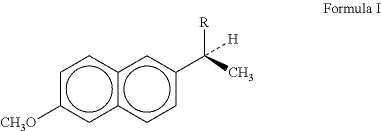

Naproxen (R═—COOH)

Naproxen sodium (R═—COONa)

Naproxen is a non-steroidal anti-inflammatory drug (NSAID) commonly used for the reduction of moderate to severe pain, fever, inflammation and stiffness caused by conditions such as osteoarthritis, rheumatoid arthritis, psoriatic arthritis, gout, ankylosing spondylitis, menstrual cramps, tendinitis, bursitis, and the treatment of primary dysmenorrhea. It works by inhibiting both the COX-1 and COX-2 enzymes. Naproxen has a pH dependent solubility i.e. slightly soluble in cidic pH and freely soluble in alkaline pH. It is BCS (Biopharmaceutic classification system) class II drug (low solubility and high permeability).

The pH independent agent or pH independent polymer is selected from the group comprising of hydroxypropyl methylcellulose (HPMC), hydroxypropyl cellulose (HPC), polyvinylpyrrolidone (PVP), methylcellulose, guar gum, xanthan gum, gum arabic, hydroxyethyl cellulose and ethyl acrylate and methyl methacrylate copolymer dispersion (Eudragit® NE 30 D), ethyl cellulose, polyvinyl acetate dispersion (Kollicoat® SR 30D) or combinations thereof and other such materials known to those of ordinary skill in the art.

The hydrophilic agent or swellable polymer is selected from the group comprising of polyethylene oxide, cellulose ethers, guar, guar derivatives, locust bean gum, psyllium, gum arabic, gum ghatti, gum karaya, gum tragacanth, carrageenan, agar, alginates, xanthan, scleroglucan, dextran, pectin, starch, chitin and chitosan, hydroxyethyl cellulose (HEC), hydroxypropyl cellulose (HPC), carboxymethyl cellulose (CMC), carboxymethylhydroxyethyl cellulose (CMHEC), hydroxypropylhydroxyethyl cellulose (HPHEC), methyl cellulose (MC), methylhydroxypropyl cellulose (MHPC), methylhydroxyethyl cellulose (MHEC), carboxymethylmethyl cellulose (CMMC), hydrophobically modified carboxymethyl cellulose (HMCMC) or combinations thereof and other such materials known to those of ordinary skill in the art.

According to another embodiment of the present invention, a chronotherapeutic pharmaceutical composition comprises of at least one active ingredient, a pH independent agent or pH independent polymer and hydrophilic agent. Only the active ingredient is coated with the pH independent polymer. The concentration of the active ingredient is 1 mg to 1000 mg. The composition provides an initial lag time of up to 4-6 hours followed by controlled release of the active ingredient up to 24 hours. The composition further comprises of an enteric coating. The enteric coating is done by enteric coating polymers, which allow further delay in the release of the active ingredient. The pH dependent polymers are selected from the group of shellac, methacrylic acid copolymers, (Eudragit® S or L) cellulose acetate phthalate, hydroxypropylmethylcellulose phthalate, hydroxypropylmethylcellulose acetate succinate, cellulose acetate trimellitate and polyvinyl acetate phthalate (Opadry® enteric white OY-P-7171), or combinations thereof and other such materials known to those of ordinary skill in the art.

According to another embodiment of the present invention, a process to prepare a chronotherapeutic pharmaceutical composition, which comprises of an active ingredient coated with a pH independent agent and hydrophilic agent is provided. The process comprises the steps of coating the active ingredient with pH independent agent. Coating of the active ingredient is carried out in a fluidized bed processor. The coated active ingredients are then blended with swellable and rapidly gelling hydrophilic agents. The blended composition is then compressed into tablets. The compressed tablets are then further enterically coated with enteric coating polymers to provide the chronotherapeutic pharmaceutical composition.

According to another embodiment of the present invention, the chronotherapeutic composition further comprises of pharmaceutically acceptable excipients.

Another embodiment of the present invention relates to the use of the chronotherapeutic composition for the treatment of diseases that show chronopharmacological dependence. The diseases are arthritis, gastrosophageal reflux disease, bronchial asthma, myocardial infarction, angina pectoris, hypertension.

Another embodiment of the present invention relates to a method for treatment of diseases that show chronopharmacological dependence comprising administering therapeutically effective amount of the composition to the subject.

The pharmaceutical composition is provided in a tablet form and is orally administered once a day. The active ingredients in the tablet are either in pellet, and/or granule form before compression. Various other dosage forms are possible for the composition; it could also be in the form of a capsule filled with granules or minitablets.

The technology provides two approaches i) Initial delayed release i.e. lag time up to 4-6 hours ii) Followed by controlled drug release up to 24 hours.

In the present invention the active ingredient is coated and mixed with the matrix forming hydrophilic agent and compressed into tablets. The compressed tablets are then further enteric coated with delayed release pH dependent agents. The chronotherapeutic composition contains two coatings one on the active ingredient and the other on the compressed tablet. When the particulate coated drug or coated active ingredient is compressed with matrix forming hydrophilic agent, the drug release from such system occurs through particulate coating and then through matrix around the coated particles. The hydrophilic agents provide additional barrier to get uniform and extended lag time. This is the advantage of the invention wherein, biphasic drug release path along with delayed release coating provides effective delay in drug release by preventing premature release of drug from the system. The system provides drug release and hence reduced variation in plasma drug profile between the individual subjects. The composition is a dual controlled release system thus providing the required lag time and controlled release of the active ingredient. The process for preparing such compositions is simple and cost effective.

The chronotherapeutic pharmaceutical composition and a process to prepare them have further been explicated in the examples of the invention.

### DEFINITIONS OF THE TERMS

The term “delayed release” as used herein means the release of active ingredient is delayed for 4-6 hours (lag time) and where drug release should be less than 10% of label claim.

The term “active ingredient” as used herein is from class Non-Steroidal Anti-Inflammatory Drug (NSAID).

The term “excipients” as used herein means a component of a pharmaceutical product that is not an active ingredient for example, fillers, diluents, carriers, alkalinizer, plasticizer, antiadherents, glidants, binders, solvents and the like. The excipients that are useful in preparing a pharmaceutical composition are safe, non-toxic and are acceptable for pharmaceutical use.

The term “diluent” or “filler” as used herein means inert substances used as fillers to create the desired bulk, flow properties. Such compounds include, by way of example and without limitation, dibasic calcium phosphate, microcrystalline cellulose, mannitol, pregelatinized starch, sucrose, powdered cellulose, precipitated calcium carbonate, starch, lactose, glucose and combinations thereof and other such materials known to those skilled in the art.

The term “binder” as used herein means agents used while making granules of the active ingredient by mixing it with diluent/filler. Such compounds include, by way of example and without limitation, polyvinyl pyrrolidone, hydroxypropyl cellulose (HPC), pregelatinized starch, starch, hydroxyl propyl methyl cellulose (HPMC), crospovidone and hydroxy ethyl cellulose (HEC) and combinations thereof and other such materials known to those skilled in the art.

The term “glidant” as used herein means agents used in formulations to improve flow-properties. Such compounds include, by way of example and without limitation, colloidal silicon dioxide, calcium silicate, magnesium silicate, cornstarch, talc, combinations thereof and other such materials known to those skilled in the art.

The term “pH independent agent” or “pH independent polymer” as used herein means polymers which shows similar change throughout all pH range i.e. it doesn't show any specific change in specific pH range.

The term “hydrophilic agent” or “swellable polymers” as used herein means polymers, which have pronounced affinity due to their chemical structures for aqueous solutions, in which they swell rather than dissolve.

The term “enteric coating polymers” as used herein means polymers, used to define a “pH dependent” coating which will resist dissolution in the acidic medium of the stomach and will dissolve in the environment of the small intestine.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

The following example is for illustrative purpose of the invention. The example should not be considered as limiting the scope of the present invention. Various modifications without deviating from the scope and gist of the present invention are possible.

### Example I

The ingredients and the mg per unit dose formula of the composition of these examples are set forth in tables below:

### Step I: Development of Naproxen Granules Using Fluidized Bed Processor (FBP)

| Ingredients | Mg per tablet |
|---|---|
| Naproxen | 500.0 |
| Dibasic calcium phosphate | 126.5 |
| dihydrate | |
| Colloidal silicon dioxide | 3.5 |
| Polyvinyl pyrrolidone K30 | 70.0 |
| Demineralised (DM) water | q.s. |

### Procedure:

1. Naproxen, Dibasic calcium phosphate dihydrate and Colloidal silicon dioxide were weighed and passed through #40 mesh American society for testing materials standards (ASTM).
2. The above blend was transferred to fluidized bed processor and mixed well for 2 minutes.
3. Required quantity of polyvinyl pyrrolidone K30 was weighed and added to DM water with continuous stirring to prepare final 25% w/v aqueous solution as binding solution.
4. Mixed blend of step 2 was granulated into fluidized bed processor by using binding solution of step 3.
5. Prepared granules were dried in fluidized bed processor to get 2-3% moisture content.

### Step II: Coating of Naproxen Granules of Step I by 30% w/w Polyacrylates Dispersion (Eudragit® NE 30D) for 5% Polymer Weight Gain Using FBP.

| Ingredients | Mg per tablet |
|---|---|
| Eudragit ® NE 30 D | 116.7 |
| Talc | 17.5 |
| DMWater | q.s |

### Procedure:

1. Required quantity of Eudragit® NE 30 D was weighed.
2. Required quantity of talc was weighed and sifted through #60 mesh (ASTM).
3. Required quantity of DM water was weighed and the talc from step 2 was added to it under stirring (avoiding foam formation).
4. Once uniform dispersion was obtained, Eudragit® NE 30 D was added slowly to the dispersion of step 3 and mixed for 30 minutes. Final dispersion contains 20% w/v solid contents.
5. The dispersion was used for coating of Naproxen granules.
6. The granules #60 mesh ASTM passed and #80 mesh ASTM retained were used for coating with Eudragit® NE 30 D (pH independent polymer).

### Step III: Compression of Naproxen Chronotherapeutic Drug Release Tablets (500 mg) and its Enteric Coating

| Ingredients | Mg per tablet |
|---|---|
| 5% w/w Eudragit ® NE 30 D | 752.5 |
| coated Naproxen granules | |
| Dibasic calcium phosphate | 116.5 |
| dihydrate | |
| Polyethylene oxide | 110.0 |
| Sodium alginate | 110.0 |
| Magnesium stearate | 11.0 |
| Uncoated tablet weight | 1100.0 |

Enteric coating solution

| | |
|---|---|
| Polyvinyl acetate phthalate | 66.0 |
| (Opadry ® enteric white OY-P- | |
| 7171) | |
| Isopropyl alcohol:methylene | q.s. |
| chloride (60:40) | |
| Enteric-coated tablet weight | 1166.0 |

### Procedure:

1. Required quantity of 5% w/w Eudragit® NE 30D coated Naproxen granules were weighed.
2. Granules of step 1 were mixed with #40 mesh ASTM passed dibasic calcium phosphate dihydrate, polyethylene oxide and sodium alginate.
3. The blend of step 2 was lubricated by magnesium stearate and compressed into tablets.
4. Compressed tablets were then enteric coated with polyvinyl acetate phthalate (Opadry® enteric white OY-P-7171).

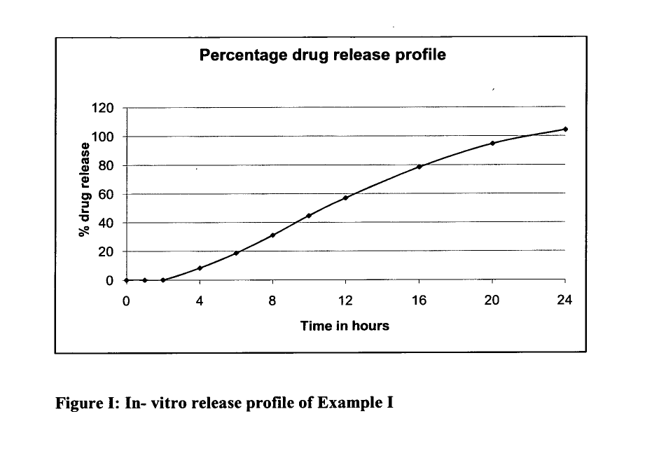

The chronotherapeutic pharmaceutical composition of Naproxen was then tested for its dissolution profile under dissolution conditions: USP Type-II, 1000 mL, 75 RPM, 0-2 Hrs. 0.1N HCl & 2-24 Hrs. Phosphate buffer pH 6.8. The dissolution profiles are set forth below in Table 1 and a graphical representation is shown in FIG. 1.

TABLE 1
Dissolution Profile

| Time (hrs) | % drug release |
|---|---|
| 1 | 0.0 |
| 2 | 0.1 |
| 4 | 8.4 |
| 6 | 18.7 |
| 8 | 31.1 |
| 10 | 44.6 |
| 12 | 57.0 |
| 16 | 78.4 |
| 20 | 94.7 |
| 24 | 104.3 |

1. A chronotherapeutic pharmaceutical composition, the composition comprising
at least one active ingredient;
a pH independent agent for coating the active ingredient; and
a hydrophilic agent for forming matrix around the coated active ingredient;
wherein the composition provides an initial lag time of 4 to 6 hours followed by controlled release of the active ingredient over a period of 24 hours.

2. The composition as claimed in claim 1, wherein the active ingredient is from the class of NSAIDs.

3. The composition as claimed in claim 4, wherein NSAIDs are selected from naproxen, lornoxicam, dilcofenac, ibuprofen and salts thereof.

4. The composition as claimed in claim 5, wherein the preferable NSAID is naproxen sodium.

5. The composition as claimed in claim 1, wherein the pH independent agent is selected from hydroxypropyl methylcellulose (HPMC), hydroxypropyl cellulose (HPC), polyvinylpyrrolidone (PVP), methylcellulose, guar gum, xanthan gum, gum arabic, hydroxyethyl cellulose and ethyl acrylate and methyl methacrylate copolymer dispersion or combinations thereof.

6. The composition as claimed in claim 1, wherein the hydrophilic agent is selected from polyethylene oxide, cellulose ethers, guar, guar derivatives, locust bean gum, psyllium, gum arabic, gum ghatti, gum karaya, gum tragacanth, carrageenan, agar, alginates, xanthan, scleroglucan, dextran, pectin, starch, chitin and chitosan, hydroxyethyl cellulose (HEC), hydroxypropyl cellulose (HPC), carboxymethyl cellulose (CMC), carboxymethylhydroxyethyl cellulose (CMHEC), hydroxypropylhydroxyethyl cellulose (HPHEC), methyl cellulose (MC), methylhydroxypropyl cellulose (MHPC), methylhydroxyethyl cellulose (MHEC), carboxymethylmethyl cellulose (CMMC), hydrophobically modified carboxymethyl cellulose (HMCMC), ethyl cellulose, polyvinyl acetate dispersion or combinations thereof.

7. The composition as claimed in claim 1, wherein the composition further comprises an enteric coating.

8. The composition as claimed in claim 9, wherein the enteric coating is of pH dependent polymer.

9. The composition as claimed in claim 10, wherein the pH dependent polymer is selected from the group of shellac, methacrylic acid copolymers, cellulose acetate phthalate, hydroxypropylmethylcellulose phthalate, hydroxypropylmethylcellulose acetate succinate, cellulose acetate trimellitate and polyvinyl acetate phthalate or combinations thereof.

10. The composition as claimed in claim 1, wherein the composition is in the form of tablets, granules or capsules.

11. The composition as claimed in claim 1, wherein the composition further comprises pharmaceutically known excipients.

12. The composition as claimed in claim 1, wherein the concentration of active ingredient is 1 mg to 1000 mg.

13. A process for preparing a tablet dosage form from the chronotherapeutic composition of any one of the claims 1-12, the process comprising the steps of:
coating the active ingredients with pH independent agent;
blending the coated active ingredient with hydrophilic agent; and
compressing the blend of coated active ingredient with hydrophilic agent into tablets.

14. The process as claimed in claim 13 further comprises enteric coating of the compressed tablet.

15. The composition as claimed in claim 1, wherein the composition is used for the treatment of diseases showing chronopharmacological dependence.

16. The composition as claimed in claim 1, wherein the diseases are arthritis, gastrosophageal reflux disease, bronchial asthma, myocardial infarction, angina pectoris, hypertension.

17. A method for the treatment of diseases that show chronopharmacological dependence comprising administering therapeutically effective amount of the composition of claim 1.
